(19) United States
(12) Patent Application Publication
(10) Pub. No.: US 2025/0023099 A1
(43) Pub. Date: Jan. 16, 2025

(54) ADDITIVES FOR ELECTROLYTES IN Li-ION BATTERIES

(71) Applicants: HYDRO-QUÉBEC, Montréal (CA); MURATA MANUFACTURING CO., LTD., Nagaokakyo-shi (JP)

(72) Inventors: Charlotte MALLET, Montréal (CA); Sylviane ROCHON, Saint-Adélphe (CA); Karim ZAGHIB, Longueuil (CA)

(73) Assignees: HYDRO-QUÉBEC, Montréal (CA); MURATA MANUFACTURING CO., LTD., Nagaokakyo-shi (JP)

(21) Appl. No.: 18/893,679

(22) Filed: Sep. 23, 2024

Related U.S. Application Data
(63) Continuation of application No. 17/281,120, filed on Mar. 29, 2021, now Pat. No. 12,100,807.
(60) Provisional application No. 62/741,275, filed on Oct. 4, 2018.

(51) Int. Cl. H01M 10/0567; C07C 255/50; C07C 255/51; C07C 255/55; C07D 213/57; H01M 10/0525; H01M 10/42

(52) U.S. Cl. CPC H01M 10/0567; C07C 255/50; C07C 255/51; C07C 255/55; C07D 213/57; H01M 10/0525; H01M 10/4235

(57) ABSTRACT

Method of improving the performance and safety of a Li-ion battery. The method includes using a nitrile-based small organic compound of general formula I, V or IX outlined in the application in association with the electrolyte of the battery. An electrolyte including a nitrile-based small organic compound. A battery including the electrolyte.

### RELATED APPLICATIONS

This application is a continuation of U.S. application Ser. No. 17/281,120, filed Mar. 29, 2021, titled “ADDITIVES FOR ELECTROLYTES IN Li-ION BATTERIES”, which is a U.S. National Phase Under U.S.C. § 371 of International Application No. PCT/CA2019/051415, titled “ADDITIVES FOR ELECTROLYTES IN Li-ION BATTERIES,” filed Oct. 3, 2019, which claims priority, under the applicable law, to U.S. Provisional Application No. 62/741,275, filed on Oct. 4, 2018, the contents of which are incorporated herein by reference in their entirety for all purposes.

### FIELD OF THE INVENTION

The present invention relates generally to additives for Li-ion batteries. More specifically, the present invention relates to nitrile-based additives for use in association with the electrolyte in Li-ion batteries.

### BACKGROUND OF THE INVENTION

Li-ion batteries are widely used as energy source, and the demand is increasing. Typically, such battery comprises a negative electrode or anode, a positive electrode or cathode, and an electrolyte provided between the two spaced-apart electrodes. The electrolyte may comprise organic molecules or polymers and generally also comprises a lithium salt such as LiPF₆, LiTFSI or LiFSI. Moreover, the electrolyte may comprise linear carbonates such as dimethyl carbonate (DMC), diethyl carbonate (DEC), ethylmethyl carbonate (EMC) or cyclic carbonates such as ethylene carbonate (EC), propylene carbonate (PC) and butylene carbonate (BC).

Various studies related to the nature and composition of electrolytes and aimed at improving the performance and safety of Li-ion batteries, are reported in the art. For example, the use of additives comprising one or more nitrile groups is reported [1-3]. Indeed, it is known in the art that organic compounds comprising nitrile groups present good electrochemical properties and stability at high voltage and temperature.

There is still a need for methods of improving the performance and safety of Li-ion batteries. In particular, there is a need for nitrile-based organic compounds for use as additives in electrolytes.

### SUMMARY OF THE INVENTION

The inventors have designed and prepared an additive for use in association with the electrolyte in a Li-ion battery. The additive of the invention is an organic compound as described herein below and which comprises at least one nitrile group. The organic compound is compatible with the electrolyte as well as other components of the battery.

The invention thus provides the following in accordance with aspects thereof:
(1) A method of improving the performance and safety of a Li-ion battery, comprising using a nitrile-based organic compound in association with the electrolyte of the battery, wherein the compound has a general formula I outlined below

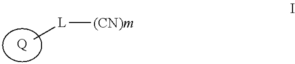

wherein:
   Q is a 5 to 12-member ring or bicycle ring, optionally the ring comprises one or more heteroatom which are the same or different and selected from the group consisting of N, O and S; preferably Q is a 5-10-, or a 5-, or a 6-member ring or bicycle ring;
   L is present or absent and is a linker comprising one or more of alkyl, alkene and alkyne groups; and
   m in an integer from 1 to 10, or 1 to 6, or I to 5, or 1 to 4, or 1 to 3.
(2) A method of improving the performance and safety of a Li-ion battery, comprising using a nitrile-based organic compound in association with the electrolyte of the battery, wherein the compound has a general formula Il outlined below

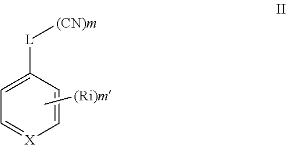

wherein:
   X is C or N;
   L is present or absent and is a linker comprising one or more of alkyl, alkene and alkyne groups;
   Ri each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably selected from the group consisting of H, alkyloxy, halogen, halogeno alkyl, nitro, and cyano; more preferably selected from the group consisting of H, halogen, nitro and cyano;
   m is an integer from 1 to 5, or 1 to 4, or 1 to 3; and
   m′ is an integer from 0 to 5, or 0 to 4, or 0 to 3, or 1 to 5, or 1 to 4, or 1 to 3.
(3) A method of improving the performance and safety of a Li-ion battery, comprising using a nitrile-based organic compound in association with the electrolyte of the battery, wherein the compound has a general formula III outlined below

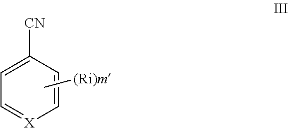

wherein:
   X is C or N;
   Ri are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably Ri are each independently selected from the group consisting of H, alkyloxy, halogen, halogeno alkyl, nitro, and cyano; more preferably selected from the group consisting of H, halogen, nitro and cyano; and
   m′ is an integer from 0 to 5, or 0 to 4, or 0 to 3, or 1 to 5, or 1 to 4, or 1 to 3.
(4) A method of improving the performance and safety of a Li-ion battery, comprising using a nitrile-based organic compound in association with the electrolyte of the battery, wherein the compound has a general formula IV outlined below

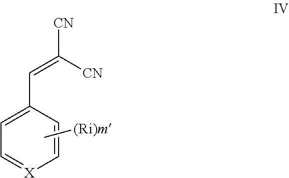

wherein:
   X is C or N;
   Ri are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably selected from the group consisting of H, alkyloxy, halogen, halogeno alkyl, nitro and cyano; more preferably selected from the group consisting of H, halogen, nitro and cyano, and
   m′ is an integer from 0 to 5, or 0 to 4, or 0 to 3, or 1 to 5, or 1 to 4, or 1 to 3.
(5) A method of improving the performance and safety of a Li-ion battery, comprising using a nitrile-based organic compound in association with the electrolyte of the battery, wherein the compound has a general formula A outlined below

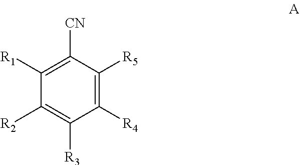

wherein: R₁ to R₅ are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably R₁ to R₅ are each independently selected from the group consisting of H, alkyloxy, halogen, halogeno alkyl, nitro and cyano; more preferably selected from the group consisting of H, halogen, nitro and cyano.
(6) A method of improving the performance and safety of a Li-ion battery, comprising using a nitrile-based organic compound in association with the electrolyte of the battery, wherein the compound has a general formula B outlined below

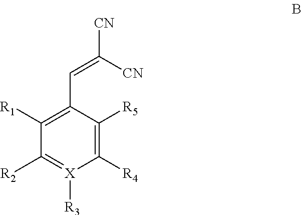

wherein:
   X is C and R₃ is H; or X is N; and
   R₁ to R₅ are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably R₁ to R₅ are each independently selected from the group consisting of H, alkyloxy, halogen, halogeno alkyl, nitro and cyano; more preferably selected from the group consisting of H, halogen, nitro and cyano.
(7) A method of improving the performance and safety of a Li-ion battery, comprising using a nitrile-based organic compound in association with the electrolyte of the battery, wherein the compound is A1, A2, A3 or A4 outlined below

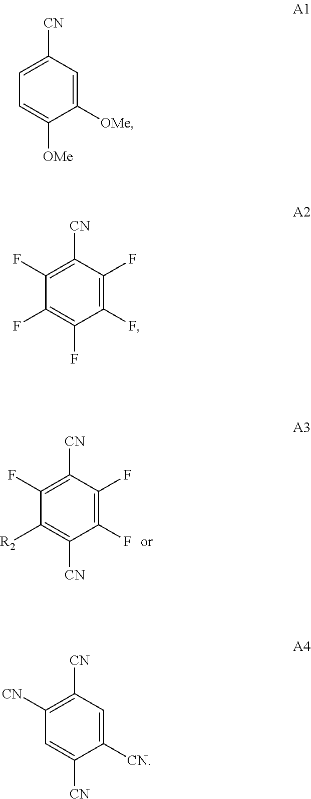

(8) A method of improving the performance and safety of a Li-ion battery, comprising using a nitrile-based organic compound in association with the electrolyte of the battery, wherein the compound is B1, B2, B3, B4, B5, B6, B7 or B8 outlined below

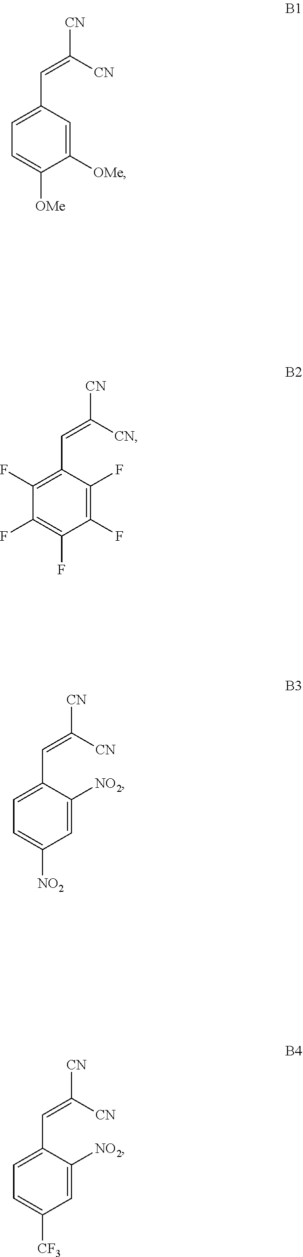 [SMILES: COc1ccc(C=C(C#N)C#N)cc1OC ; N#CC(C#N)=Cc1c(F)c(F)c(F)c(F)c1F ; N#CC(C#N)=Cc1ccc([N+](=O)[O-])cc1[N+](=O)[O-] ; N#CC(C#N)=Cc1ccc(C(F)(F)F)cc1[N+](=O)[O-]]

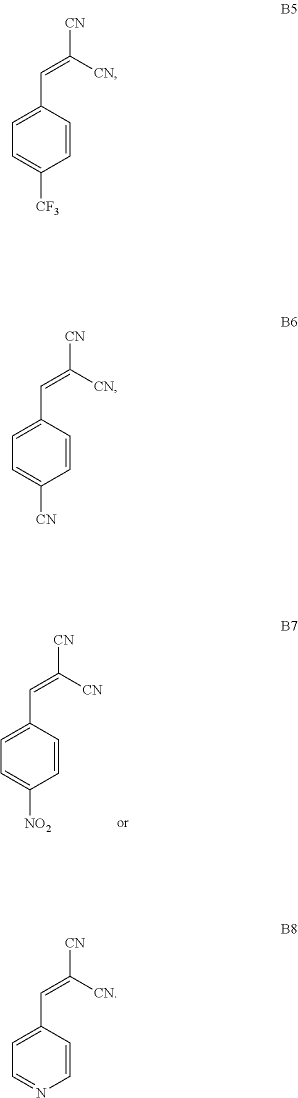 [SMILES: N#CC(C#N)=Cc1ccc(C(F)(F)F)cc1 ; N#CC(C#N)=Cc1ccc(C#N)cc1 ; N#CC(C#N)=Cc1ccc([N+](=O)[O-])cc1 ; N#CC(C#N)=Cc1ccncc1]

(9) A method of improving the performance and safety of a Li-ion battery, comprising using a nitrile-based organic compound in association with the electrolyte of the battery, wherein the compound has a general formula V outlined below

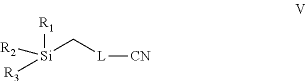

wherein:
   L is present or absent and is a linker comprising one of more of alkyl, alkene and alkyne groups; and
   R₁ to R₃ are each independently alkyl groups, preferably C1 to C6 or C1 to C3 alkyl groups; more preferably at least one of R₁ to R₃ is CH₃, or each of R₁ to R₃ is CH₃.
(10) A method of improving the performance and safety of a Li-ion battery, comprising using a nitrile-based organic compound in association with the electrolyte of the battery, wherein the compound has a general formula VI outlined below

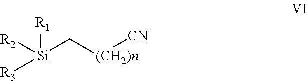

wherein:
   n is an integer from 0 to 6, or 0 to 5, or 0 to 4, or 0 to 3, or 0 to 2; preferably n is an integer from 0 to 3; more preferably n is 0 or 1; and
   R₁ to R₃ are each independently alkyl groups; preferably C1 to C6 or C1 to C3 alkyl groups, more preferably at least one of R₁ to R₃ is CH₃, or each of R₁ to R₃ is CH₃.
(11) A method of improving the performance and safety of a Li-ion battery, comprising using a nitrile-based organic compound in association with the electrolyte of the battery, wherein the compound bas a general formula C outlined below

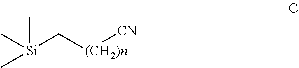

wherein n is an integer from 0 to 6, or 0 to 5, or 0 to 4, or 0 to 3, or 0 to 2; preferably n is an integer from 0 to 3; more preferably n is 0 or 1.
(12) A method of improving the performance and safety of a Li-ion battery, comprising using a nitrile-based organic compound in association with the electrolyte of the battery, wherein the compound is C1 or C2 outlined below

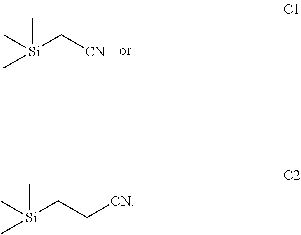

(13) A method of improving the performance and safety of a Li-ion battery, comprising using a nitrile-based organic compound in association with the electrolyte of the battery, wherein the compound bas a general formula IX outlined below

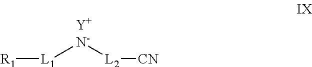

wherein:
   R₁ is CN or CH₃;
   L₁ and L₂ are each independently present or absent and are each independently a linker comprising alkyl, alkene and/or alkyne groups; and
   Y is Na, K or Li, preferably Y is Na.
(14) A method of improving the performance and safety of a Li-ion battery, comprising using a nitrile-based organic compound in association with the electrolyte of the battery, wherein the compound has a general formula X outlined below

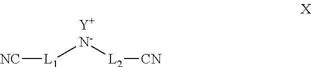

wherein:
   L₁ and L₂ are each independently present or absent and are each independently a linker comprising one or more of alkyl, alkene and alkyne groups; and Y is Na, K or Li; preferably Y is Na.
(15) A method of improving the performance and safety of a Li-ion battery, comprising using a nitrile-based organic compound in association with the electrolyte of the battery, wherein the compound has a general formula XI outlined below

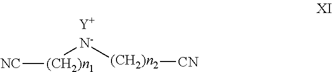

wherein:
   n1 and n2 are each independently an integer from 0 to 10, or 0 to 6, or 0 to 3;
   preferably at least one of n1 and n2 is 0, or both n1 and n2 are 0; and
   Y is Na, K or Li; preferably Y is Na.
(16) A method of improving the performance and safety of a Li-ion battery, comprising using a nitrile-based organic compound in association with the electrolyte of the battery, wherein the compound has a general formula D outlined below

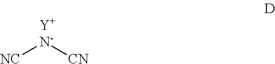

wherein Y is Na, K or Li; preferably Y is Na.
(17) A method of improving the performance and safety of a Li-ion battery, comprising using a nitrile-based organic compound in association with the electrolyte of the battery, wherein the compound has a general formula D1 outlined below

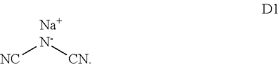

(18) A compound having a general formula VII outlined below

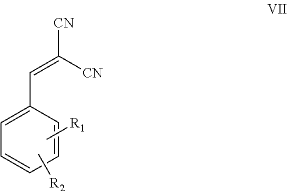

wherein R₁ and R₂ are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably selected from the group consisting of H, alkyloxy, halogen, halogeno alkyl, nitro and cyano; more preferably selected from the group consisting of H, halogen, nitro and cyano.
(19) A compound having a general formula VIII outlined below

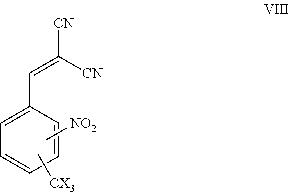

wherein X is a halogen atom; preferably X is F.
(20) A compound of formula B4 outlined below

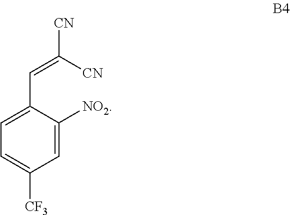 [SMILES: N#CC(C#N)=Cc1ccc(C(F)(F)F)cc1[N+](=O)[O-]]

(21) A method of improving the performance and safety of a Li-ion battery, comprising using a nitrile-based organic compound in association with the electrolyte of the battery, wherein the compound as defined in any one of (18) to (20) above.
(22) The method according to any one of (1) to (17) and (21) above, wherein the nitrile-based organic compound is added to the electrolyte; optionally an amount of the additive (nitrile-based organic compound) is between about 0.01 to about 5.0% wt, or about 0.01 to about 3.0% wt, or about 0.01 to about 1.0% wt, or about 0.05 to about 1.0% wt, or about 0.1to about 1.0% wt, about 0.1 to about 0.8% wt, or about 0.1 to about 0.5% wt, or about 0.1 to about 0.3% wt, is 0.1% wt, or is 0.5% wt.
(23) An electrolyte comprising a compound which is selected from the group consisting of: I, II, III, IV, A, B, A1, A2, A3, A4, B1, B2, B3, B4, B5, B6, B7. B8, V, VI, C, C1, C2, IX, X, XI, D, and D1 as defined in any one of the methods of (1) to (17) above.
(24) An electrolyte comprising the compound as defined in any one of (18) to (20) above.
(25) A battery comprising the electrolyte as defined in (23) or (24) above.
(26) An additive for an electrolyte for use in a Li-ion battery, comprising a compound which is selected from the group consisting of: I, II, III, IV, A, B, A1, A2, A3, A4, B1, B2,B3, B4, B5, B6, B7, B8, V, VI, C, C1, C2, IX, X, XI, D, and D1 as defined in any one of the methods of (1) to (17) above.
(27) An additive for an electrolyte for use in a Li-ion battery, comprising a compound as defined in any one of (18) to (20) above.
(28) The method, electrolyte, battery or additive according to any one of (1) to (27) above, wherein the Li-ion battery is a battery wherein the cathode comprises a lithium-containing material;
(29) The method, electrolyte, battery or additive according to any one of (1) to (27) above, wherein the Li-ion battery is a battery wherein the cathode comprises lithium cobalt oxide (LCO), lithium manganese oxide (LMO), lithium nickel oxide (LNO) and the like including olivines, lithium oxides, nickel manganese cobalt oxide (NMC).
(30) The method, electrolyte, battery or additive according to (28) or (30) above, wherein the performance (capacity, reversibility) of the battery is improved.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the appended drawings:

FIG. 1: Cycling data of LMFP-LTO battery (PC/EMC/DMC (4/3/3)+1M LiPF₆+0.1 wt % additive according to the invention (a compounds of Serie A)) versus Reference after 300 cycles at 45° C.

FIG. 2: Static capacity (0.05 C) of LMFP-LTO battery (PC/EMC/DMC (4/3/3)+1M LiPF₆+0.1 wt % additive according to the invention (a compound of Serie A)) versus Reference at 45° C.

FIG. 3: Nyquist plots of LMFP-LTO battery (PC/EMC/DMC (4/3/3)+1M LiPF₆+0.1 wt % additive according to the invention (a compound of Serie A)) versus Reference, at 0 and 100 cycles

FIG. 4: Cycling data of LMFP-LTO battery (PC/EMC/DMC (4/3/3)+1M LiPF₆+0.5 wt % additive according to the invention (a compounds of Serie B)) versus Reference after 300 cycles at 45° C.

FIG. 5: Static capacity (0.05 C) of LMFP-LTO battery (PC/EMC/DMC (4/3/3)+1M LiPF₆+0.5 wt % additive according to the invention (a compound of Serie B)) versus Reference at 45° C.

FIG. 6: Nyquist plots of LMFP-LTO battery (PC/EMC/DMC (4/3/3)+1M LiPF₆+0.5 wt % additive according to the invention (a compound of Serie B)) versus Reference, at 0 and 200 cycles.

FIG. 7: Cycling data of LMFP-LTO battery (PC/EMC/DMC (4/3/3)+1M LiPF₆+0.5 wt % additive according to the invention (a compounds of Serie C)) versus Reference after 300 cycles at 45° C.

FIG. 8: Static capacity (0.05 C) of LMFP-LTO battery (PC/EMC/DMC (4/3/3)+1M LiPF₆+0.5 wt % additive according to the invention (a compound of Serie C)) versus Reference at 45° C.

FIG. 9: Nyquist plots of LMFP-LTO battery (PC/EMC/DMC (4/3/3)+1M LiPF₆+0.5 wt % additive according to the invention (a compound of Serie C)) versus Reference, at 0 and 100 cycles.

FIG. 10: Cycling data of LMFP-LTO battery (PC/EMC/DMC (4/3/3)+1M LiPF₆+0.5 wt % additive according to the invention (a compounds of Serie D)) versus Reference after 100 cycles at 45° C.

FIG. 11: Static capacity (0.05 C) of LMFP-LTO battery (PC/EMC/DMC (4/3/3)+1M LiPF₆+0.5 wt % additive according to the invention (a compound of Serie D)) versus Reference at 45° C.

### DESCRIPTION OF ILLUSTRATIVE EMBODIMENTS

Before the present invention is further described, it is to be understood that the invention is not limited to the particular embodiments described below, as variations of these embodiments may be made and still fall within the scope of the appended claims. It is also to be understood that the terminology employed is for the purpose of describing particular embodiments, and is not intended to be limiting. Instead, the scope of the present invention will be established by the appended claims.

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this disclosure pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used herein when referring to numerical values or percentages, the term “about” includes variations due to the methods used to determine the values or percentages, statistical variance and human error. Moreover, each numerical parameter in this application should at least be construed in light of the number of reported significant digits and by applying ordinary rounding techniques.

Term “alkyl” or “alk” as used herein, represents a monovalent group derived from a straight or branched chain saturated hydrocarbon comprising, unless otherwise specified, from 1 to 15 carbon atoms and is exemplified by methyl, ethyl, n- and iso-propyl, n-, sec-, iso-and tert-butyl, neopentyl and the like and may be optionally substituted with one, two, three or, in the case of alkyl groups comprising two carbons or more, four substituents.

The term “alkoxy” or “alkyloxy” as used interchangeably herein, represents an alkyl group attached to the parent molecular group through an oxygen atom.

The term “alkylthio” or “thioalkoxy” as used interchangeably herein, represents an alkyl group attached to the parent molecular group through a sulfur atom.

The term “alkylene” as used herein, represents a saturated divalent hydrocarbon group derived from a straight or branched chain saturated hydrocarbon by the removal of two hydrogen atoms, and is exemplified by methylene, ethylene, isopropylene and the like.

The term “alkenyl” as used herein, represents monovalent straight or branched chain groups of, unless otherwise specified, from 2 to 15 carbons, such as, for example, 2 to 6 carbon atoms or 2 to 4 carbon atoms, containing one or more carbon-carbon double bonds and is exemplified by ethenyl, 1-propenyl, 2-propenyl, 2-methyl-1-propenyl, 1-butenyl, 2-butenyl and the like and may be optionally substituted with one, two, three or four substituents.

The term “alkynyl” as used herein, represents monovalent straight or branched chain groups of from two to six carbon atoms comprising a carbon-carbon triple bond and is exemplified by ethynyl, 1-propynyl, and the like and may be optionally substituted with one, two, three or four substituents.

The term “cycloalkyl” as used herein, represents a monovalent saturated or unsaturated non-aromatic cyclic hydrocarbon group of three to eight carbon atoms, unless otherwise specified, and is exemplified by cyclopropyl, cyclobutyl, cyclopentyl, cyclohexyl, cycloheptyl, bicyclo[2.2.1]heptyl and the like.

The term “halogen” or “halo” as used interchangeably herein, represents F, Cl, Br and I.

The term “heteroatom”, as used herein, is understood as being oxygen, sulfur or nitrogen.

The inventors have designed and prepared an additive for use in association with the electrolyte in a Li-ion battery. The additive of the invention is an organic compound as described herein below and which comprises at least one nitrile group. Also, the organic compound is compatible with the electrolyte as well as other components of the battery.

More specifically, the additive of the invention for use in association with the electrolyte is a nitrile-based organic compound as described herein and having general formulae I-XI, A, B, C and D depicted below.

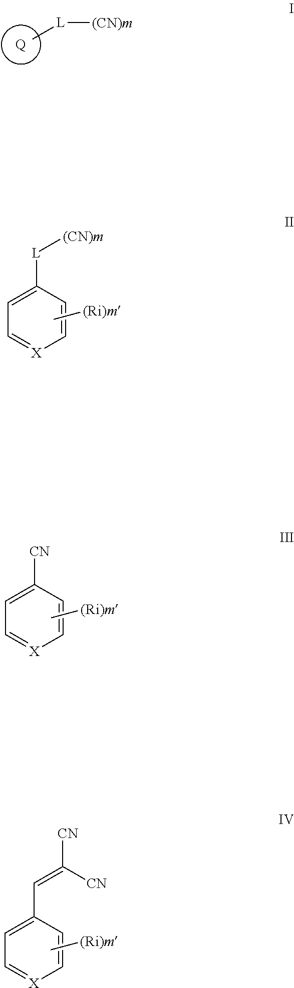

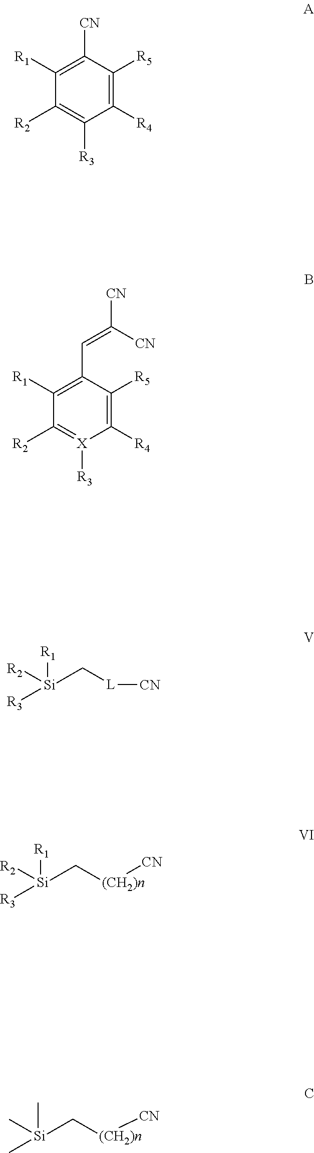

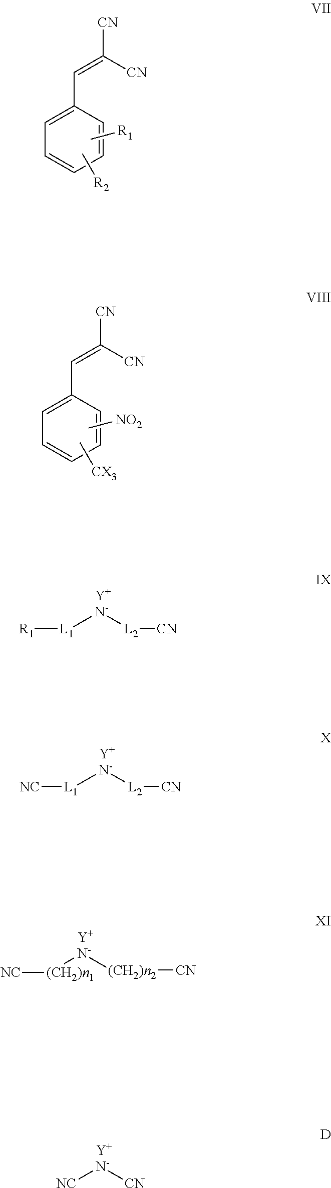

Such organic compounds are exemplified by compounds defined in Table 1 below, namely, Compounds A1-A4, B1-B8, C1-C2 and D1.

TABLE 1
Organic compounds according to the invention (Series A, B, C and D)

| | | R₁ | R₂ | R₃ | R₄ | R₅ | X | n | | Cycle |
|---|---|---|---|---|---|---|---|---|---|---|
| A | A1 | H | H | OMe | OMe | H | | | 0.1% | 300 |
| | A2 | F | F | F | F | F | | | 0.1% | 300 |
| | A3 | F | F | CN | F | F | | | 0.1% | 300 |
| | A4 | CN | H | CN | CN | H | | | 0.1% | 300 |
| B | B1 | H | H | OMe | OMe | H | C | | 0.5% | 300 |
| | B2 | F | F | F | F | F | C | | 0.5% | 200 |
| | B3 | H | H | NO₂ | H | NO₂ | C | | 0.5% | poor results |
| | B4 | H | H | CF₃ | H | NO₂ | C | | 0.5% | 300 |
| | B5 | H | H | CF₃ | H | H | C | | 0.5% | 300 |
| | B6 | H | H | CN | H | H | C | | 0.5% | 300 |
| | B7 | H | H | NO₂ | H | H | C | | 0.5% | poor results |
| | B8 | H | H | H | H | H | N | | 0.5% | 300 |
| C | C1 | | | | | | | 0 | 0.5% | 300 |
| | C2 | | | | | | | 1 | 0.5% | 300 |
| D | D1 | | | | | | | | 0.5% | 300 |

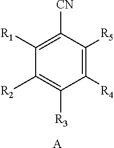

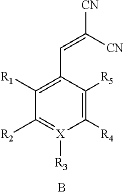

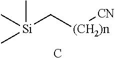

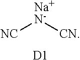

The present invention is illustrated in further details by the following non-limiting examples.

### Nitrile-Based Organic Compounds for Use as Additive in Association With Li-Ion Electrolytes

### Example 1

General Procedure for the Preparation of the Compounds. To a solution of aldehyde (1 eq.) in 15 mL of chloroform are added, molonodinitrile (1.5 eq.) and few drops of triethylamine. The mixture is refluxed one night under nitrogen. After return to room temperature, dichloromethane is added, and the solution is washed twice with water and dried over MgSO₄. After solvent removal, the residue is chromatographed (silica gel/dichloromethane) to give a solid.

### Example 2

### Compound B1

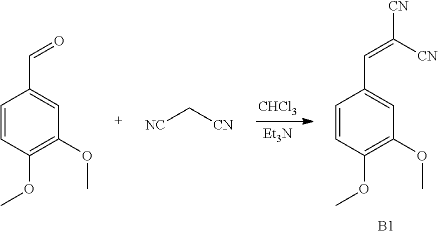

Bright yellow solid (70%). NMR ¹H (400 MHz, CDCl₃) δ: 7.69 (d, 1H, J=4 Hz); 7.64 (s, 1H); 7.38 (dd, 1H, J=4 Hz, J=12 Hz); 6.95 (d, 1H, J=12 Hz); 3.99 (s, 3H); 3.93 (s, 3H).

### Example 3

### Compound B2

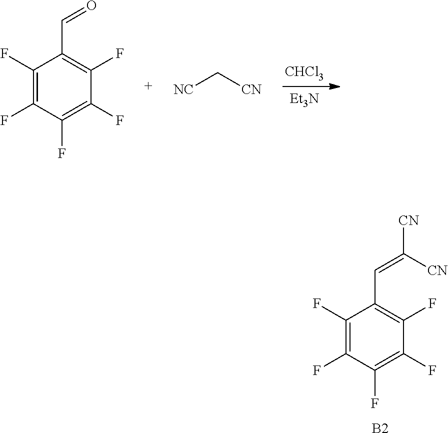

Yellow solid (40%). NMR ¹H (400 MHz, CDCl₃) δ: 7.77 (s, 1H). NMR ¹⁹F (400 MHz, CDCl₃) δ: −132.55 (s, 2H); −143.68 (s, 1H); −158.50 (s, 1H).

### Example 4

### Compound B3

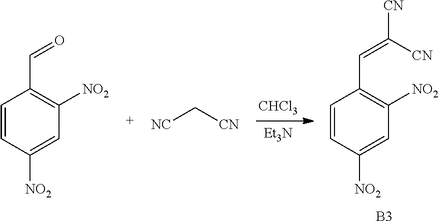

White solid. NMR ¹H (400 MHz, CDCl₃) δ: 8.60 (d, 1H, J=4 Hz); 8.25 (dd, 1H, J=4 Hz, J=12 Hz); 8.18 (s, 1H); 8.15 (d, 1H, J=12 Hz).

### Example 5

### Compound B4

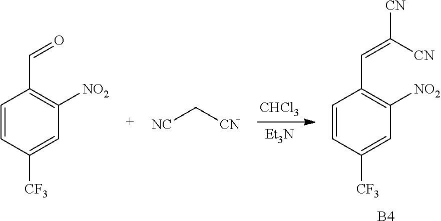

Bright yellow solid. NMR ¹H (400 MHz, CDCl₃) δ: 8.12 (d, 1H, J=4 Hz); 8.03 (s, 1H); 7.67 (dd, 1H, J=4 Hz, J=12 Hz). NMR ¹⁹F (400 MHz, CDCl₃) δ: −63.65 (s, 3F).

### Example 6

### Compound B5

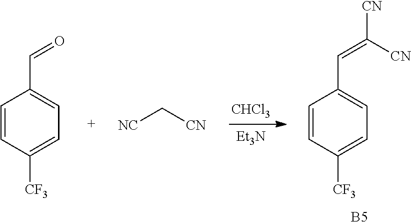

White solid. NMR ¹H (400 MHz, CDCl₃) δ: 8.02 (d, 2H, J=12 Hz); 7.83 (d, 2H, J=8 Hz); 7.80 (s, 1H). NMR ¹⁹F (400 MHz, CDCl₃) δ: −63.48 (s, 3F).

### Example 7

### Compound B6

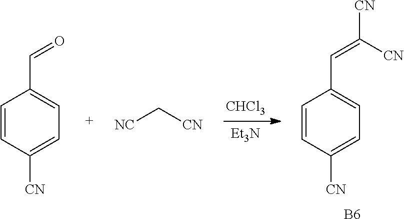

White solid. NMR ¹H (400 MHz, CDCl₃) δ: 7.99 (d, 2H, J=8 Hz); 7.83 (d, 2H, J=8 Hz); 7.74 (s, 1H).

### Example 8

### Compound B7

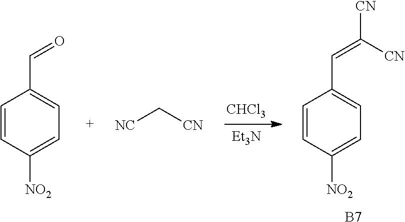

Pale orange solid. NMR ¹H (400 MHz, CDCl₃) δ: 8.39 (d, 2H, J=12 Hz); 8.07 (d, 2H, J=8 Hz); 7.88 (s, 1H).

### Example 9

### Compound B8

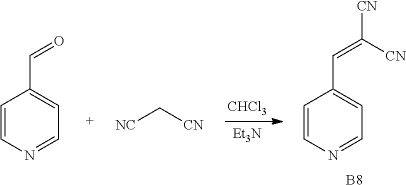

Pink solid. NMR ¹H (400 MHz, CDCl₃) δ: 8.89 (d, 2H, J=12 Hz); 7.81 (s, 2H); 7.68 (d, 2H, J=8 Hz).

Compounds of the Series A and C and Compound DI are commercially available and were used as received.

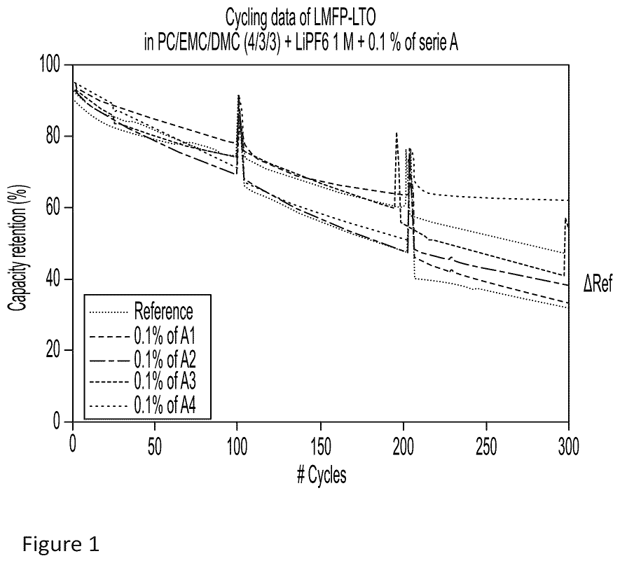

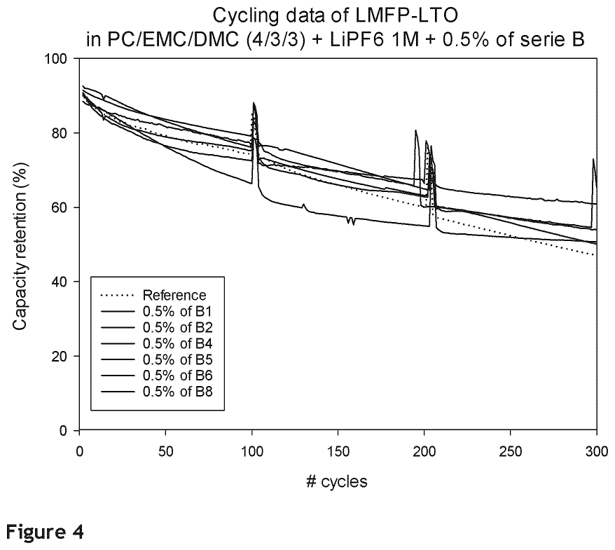

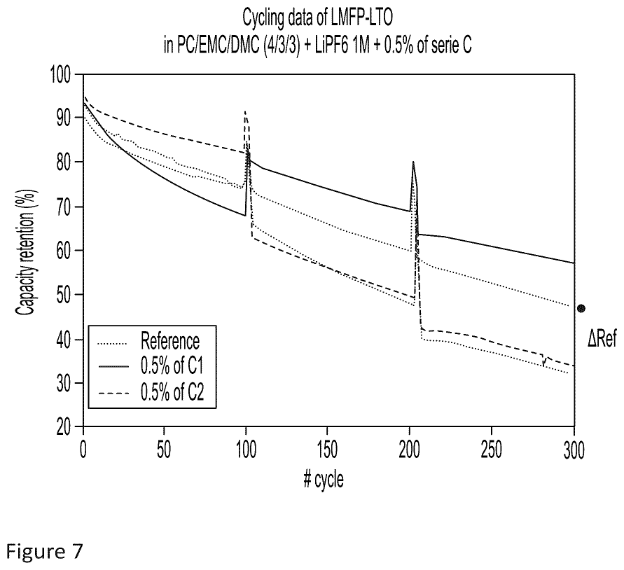

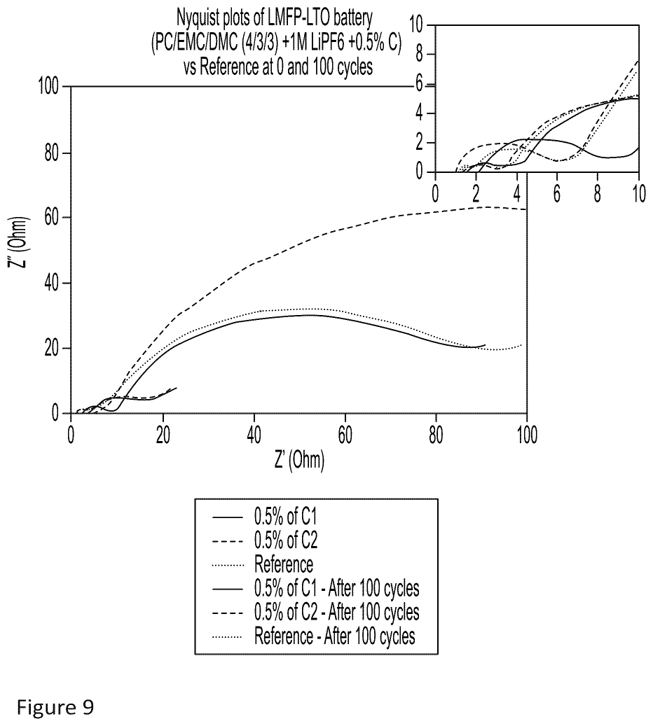

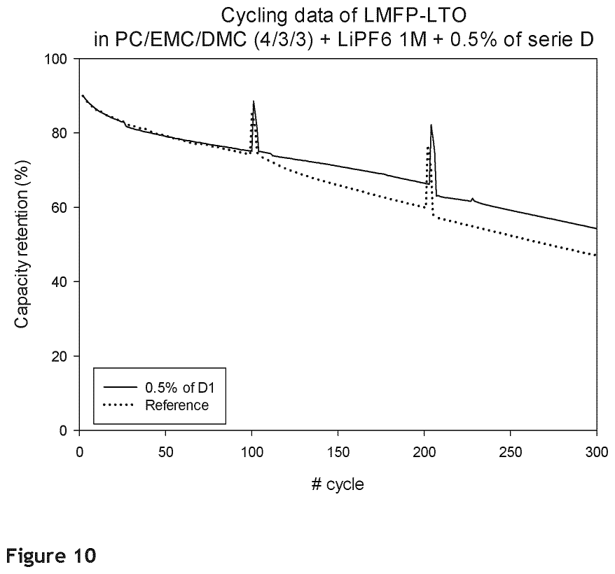

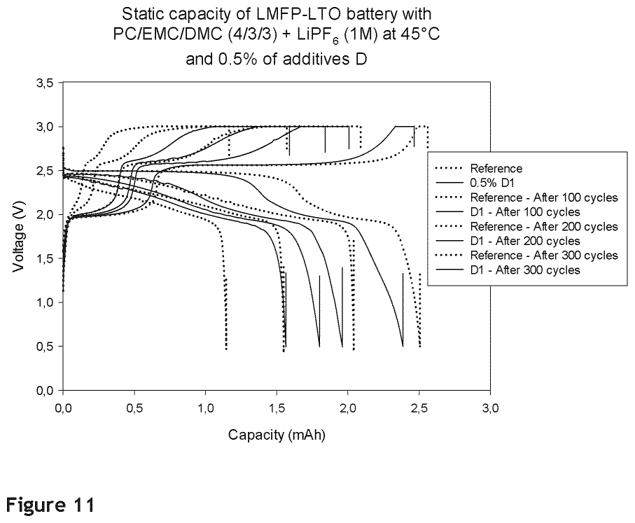

Referring to the figures, FIGS. 1-3 outline results obtained using compounds of the Serie A; FIGS. 4-6 outline results obtained using compounds of the Serie B; FIGS. 7-9 outline results obtained using compounds of the Serie C; and FIGS. 10-11 outline results obtained using compounds of the Serie D. It should be noted that Reference batteries as well as batteries according to the invention, do not contain vinylene carbonate (VC), which explains the poor stability after 300 cycles. Nonetheless as can be seen, batteries comprising the additive according to the invention present a far better stability.

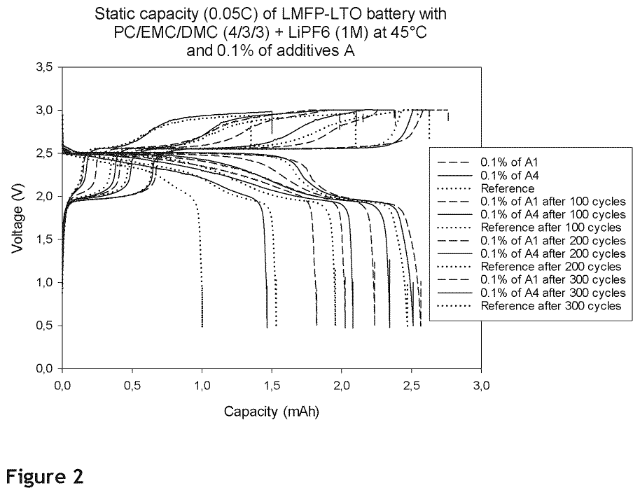

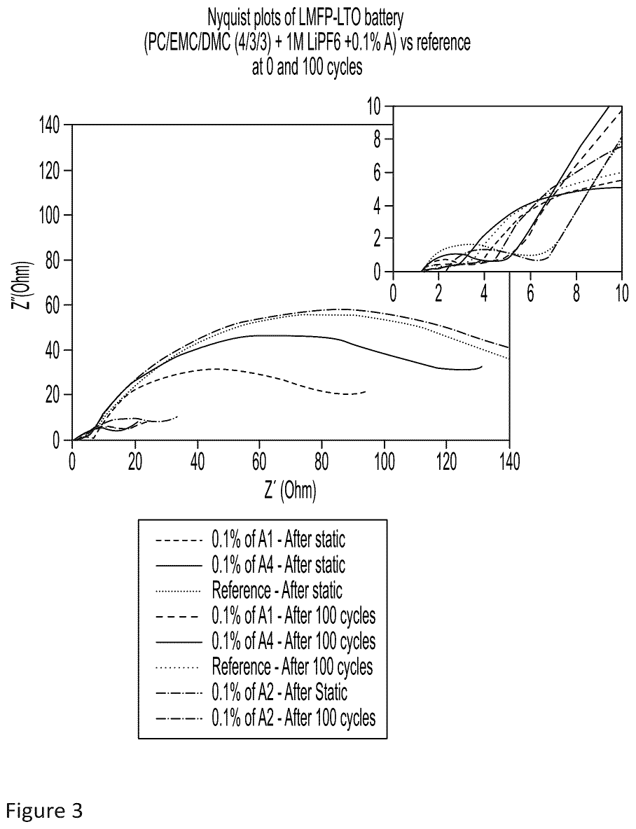

As can be seen in FIG. 2, use of 0.1 wt % of compound A1 or A4 allows for improvement of the battery capacity as well as a better reversibility. Moreover, a global decrease of the battery resistance is noted (FIG. 3).

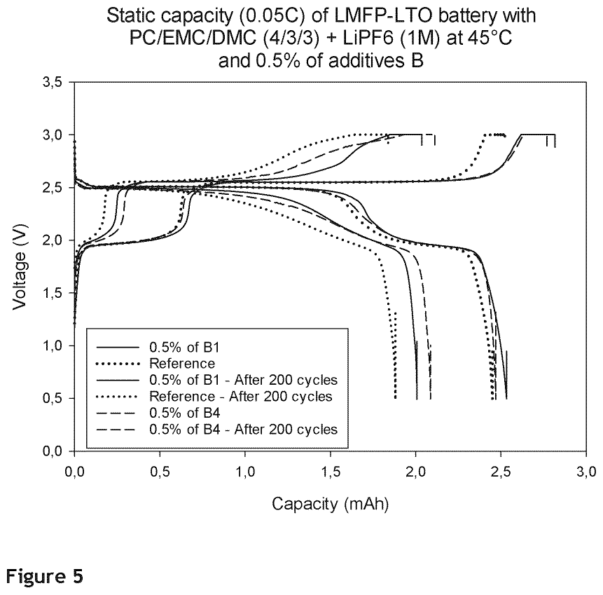

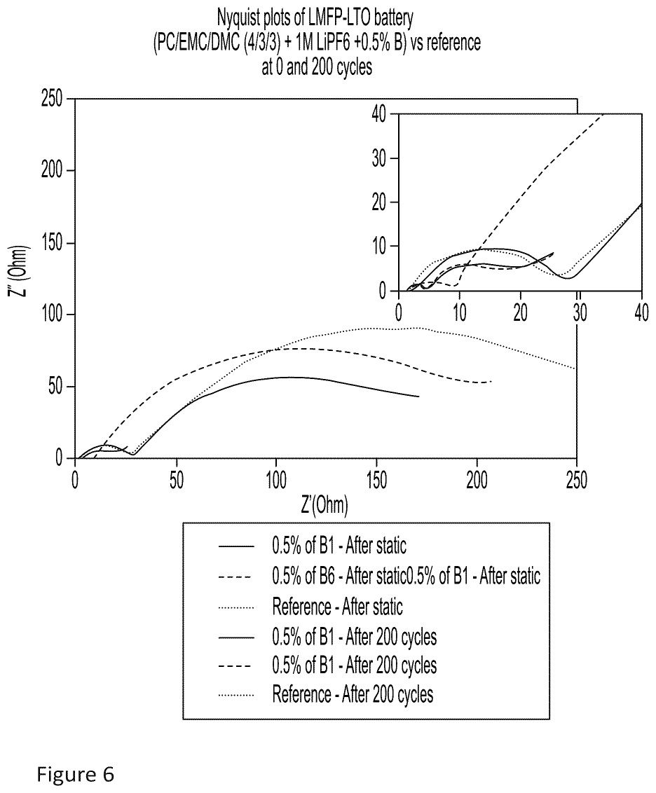

FIG. 5 shows results obtained for compounds B1 and B4. Use of 0.5 wt % of the additive allows for an improvement of the battery capacity. A global decrease of the battery resistance is noted (FIG. 6).

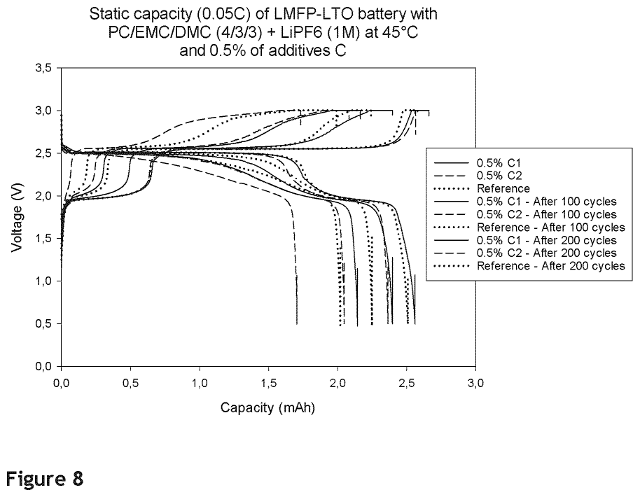

FIG. 7 shows results obtained for compounds C1 and C2. Use of 0.5 wt % of the additive yields a good stability after 300 cycles at 45° C. As can be seen in FIG. 8, better results are obtained for compound C1 (shorter carbon chain).

FIG. 10 shows results obtained for compound D1. As can be seen in FIG. 11, use of 0.5 wt % of compound D1 allows for improvement of the battery capacity as well as a better reversibility.

As will be understood by a skilled person, the additive for use in association with the electrolyte are adapted to be compatible with the components of the battery including the electrolyte and the cathode active material.

The invention is described in relation to lithium manganese iron phosphate (LMFP)—lithium titanium oxide (LTO) batteries. As will be understood by a skilled person, other lithium-ion batterie types may also be used. In other words, any battery wherein the cathode active material comprises a lithium-containing material may be used. Such lithium-containing material may be lithium cobalt oxide (LCO), lithium manganese oxide (LMO), lithium nickel oxide (LNO) and the like including olivines, lithium oxides, nickel manganese cobalt oxide (N M C).

Also, as will be understood by a skilled person, the anode material may be of any suitable type, such as for example lithium alloys, Si, SiOx, graphite and carbon mixtures, titanates, lithium titanates.

The scope of the claims should not be limited by the preferred embodiments set forth in the examples but should be given the broadest interpretation consistent with the description as a whole.

The present description refers to a number of documents, the content of which is herein incorporated by reference in their entirety.

### REFERENCES

1. Rohan R. et al. J. Phyx. Chem C (2016), 120 (12), 6450-6458.
2. Kim Y.-S. et al. ACS Appl. Mater. Interfaces (2014), 6 (11), 8913-8920.
3. Pohl B. et al. J. Electrochem. Soc. (2015), 162 (3), A460-A464.

1. A method of improving the performance and safety of a Li-ion battery, comprising using a nitrile-based organic compound in association with the electrolyte of the battery, wherein the compound has a general formula I outlined below 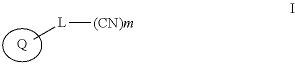
wherein:
Q is a 5 to 12-member ring or bicycle ring, optionally the ring comprises one or more heteroatom which are the same or different and selected from the group consisting of N, O and S; preferably Q is a 5-10-, or a 5-, or a 6-member ring or bicycle ring;
L is present or absent and is a linker comprising one or more of alkyl, alkene and alkyne groups; and
m in an integer from 1 to 10, or 1 to 6, or 1 to 5, or 1 to 4, or 1 to 3.

2. A method of improving the performance and safety of a Li-ion battery, comprising using a nitrile-based organic compound in association with the electrolyte of the battery, wherein the compound has a general formula II outlined below 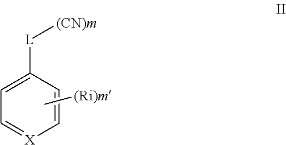
wherein:
X is C or N;
L is present or absent and is a linker comprising one or more of alkyl, alkene and alkyne groups;
Ri each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl;
preferably selected from the group consisting of H, alkyloxy, halogen, halogeno alkyl, nitro, and cyano; more preferably selected from the group consisting of H, halogen, nitro and cyano;
m is an integer from 1 to 5, or 1 to 4, or 1 to 3; and
m′ is an integer from 0 to 5, or 0 to 4, or 0 to 3, or 1 to 5, or 1 to 4, or 1 to 3.

3. A method of improving the performance and safety of a Li-ion battery, comprising using a nitrile-based organic compound in association with the electrolyte of the battery, wherein the compound has a general formula III outlined below 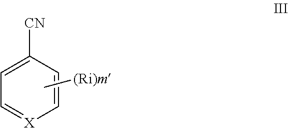
wherein:
X is C or N;
Ri are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl;
preferably Ri are each independently selected from the group consisting of H, alkyloxy, halogen, halogeno alkyl, nitro, and cyano; more preferably selected from the group consisting of H, halogen, nitro and cyano; and
m′ is an integer from 0 to 5, or 0 to 4, or 0 to 3, or 1 to 5, or 1 to 4, or 1 to 3.

4. A method of improving the performance and safety of a Li-ion battery, comprising using a nitrile-based organic compound in association with the electrolyte of the battery, wherein the compound has a general formula IV outlined below 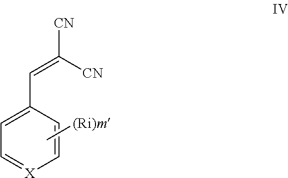
wherein:
X is C or N;
Ri are each independently selected from the group consisting of H, alkyl, cycloalkyl, alkene, alkyne, aryl and alkylaryl, alkoxy, thioalkoxy, OH, SH, NH₂, a halogen atom, a balogeno alkyl, a halogeno alkoxy, a halogeno thioalkoxy, a cyano alkyl, a cyano alkene, a cyano alkyne, CN, NO₂, SO₂, COOH and acyloxycarbonyl;
preferably selected from the group consisting of H, alkyloxy, halogen, halogeno alkyl, nitro and cyano; more preferably selected from the group consisting of H, halogen, nitro and cyano; and
m′ is an integer from 0 to 5, or 0 to 4, or 0 to 3, or 1 to 5, or 1 to 4, or 1 to 3.

5. A method of improving the performance and safety of a Li-ion battery, comprising using a nitrile-based organic compound in association with the electrolyte of the battery, wherein the compound has a general formula A outlined below 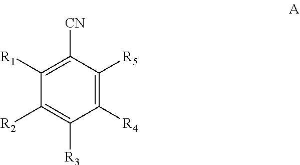
wherein: R₁ to R₅ are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably R₁ to R₅ are each independently selected from the group consisting of H, alkyloxy, halogen, halogeno alkyl, nitro and cyano; more preferably selected from the group consisting of H, halogen, nitro and cyano.

6. A method of improving the performance and safety of a Li-ion battery, comprising using a nitrile-based organic compound in association with the electrolyte of the battery, wherein the compound has a general formula B outlined below 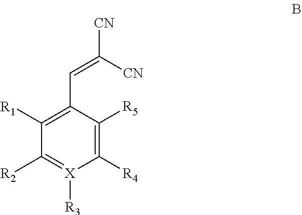
wherein:
X is C and R₃ is H; or X is N; and
R₁ to R₅ are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl;
preferably R₁ to R₅ are each independently selected from the group consisting of H, alkyloxy, halogen, halogeno alkyl, nitro and cyano; more preferably selected from the group consisting of H, halogen, nitro and cyano.

7. A method of improving the performance and safety of a Li-ion battery, comprising using a nitrile-based organic compound in association with the electrolyte of the battery, wherein the compound is A1, A2, A3 or A4 outlined below

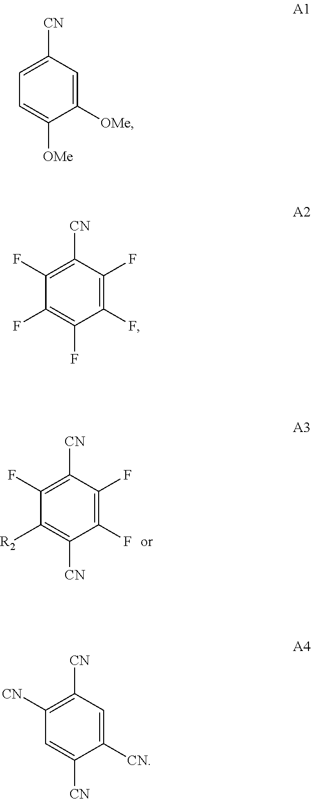

8. A method of improving the performance and safety of a Li-ion battery, comprising using a nitrile-based organic compound in association with the electrolyte of the battery, wherein the compound is B1, B2, B3, B4, B5, B6, B7 or B8 outlined below

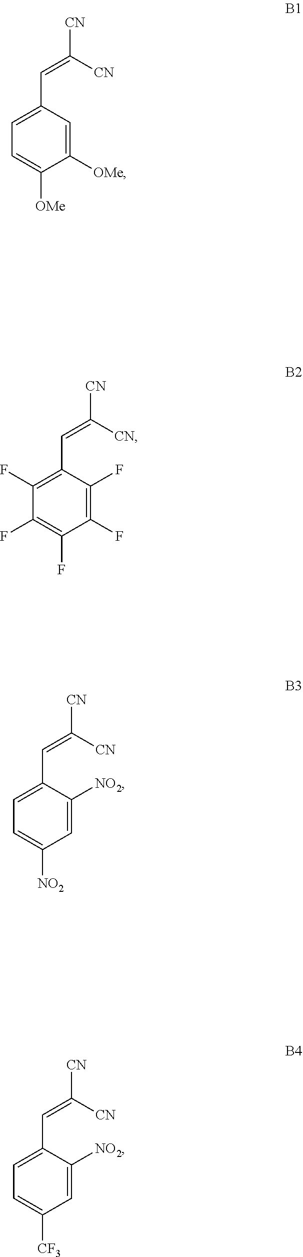 [SMILES: COc1ccc(C=C(C#N)C#N)cc1OC ; N#CC(C#N)=Cc1c(F)c(F)c(F)c(F)c1F ; N#CC(C#N)=Cc1ccc([N+](=O)[O-])cc1[N+](=O)[O-] ; N#CC(C#N)=Cc1ccc(C(F)(F)F)cc1[N+](=O)[O-]]

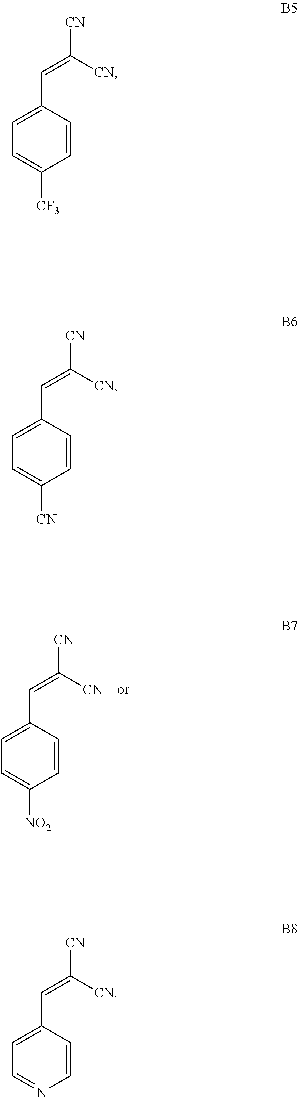 [SMILES: N#CC(C#N)=Cc1ccc(C(F)(F)F)cc1 ; N#CC(C#N)=Cc1ccc(C#N)cc1 ; N#CC(C#N)=Cc1ccc([N+](=O)[O-])cc1 ; N#CC(C#N)=Cc1ccncc1]

9. A method of improving the performance and safety of a Li-ion battery, comprising using a nitrile-based organic compound in association with the electrolyte of the battery, wherein the compound has a general formula V outlined below 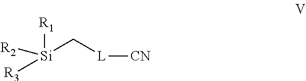
wherein:
L is present or absent and is a linker comprising one of more of alkyl, alkene and alkyne groups; and
R₁ to R₃ are each independently alkyl groups; preferably C1 to C6 or C1 to C3 alkyl groups; more preferably at least one of R₁ to R₃ is CH₃, or each of R₁ to R₃ is CH₃.

10. A method of improving the performance and safety of a Li-ion battery, comprising using a nitrile-based organic compound in association with the electrolyte of the battery, wherein the compound has a general formula VI outlined below 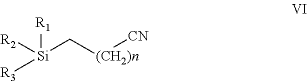
wherein:
n is an integer from 0 to 6, or 0 to 5, or 0 to 4, or 0 to 3, or 0 to 2; preferably n is an integer from 0 to 3; more preferably n is 0 or 1; and
R₁ to R₃ are each independently alkyl groups; preferably C1 to C6 or C1 to C3 alkyl groups; more preferably at least one of R₁ to R₃ is CH₃, or each of R₁ to R₃ is CH₃.

11. A method of improving the performance and safety of a Li-ion battery, comprising using a nitrile-based organic compound in association with the electrolyte of the battery, wherein the compound has a general formula C outlined below 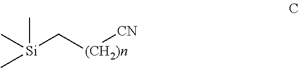
wherein n is an integer from 0 to 6, or 0 to 5, or 0 to 4, or 0 to 3, or 0 to 2; preferably n is an integer from 0 to 3; more preferably n is 0 or 1.

12. A method of improving the performance and safety of a Li-ion battery, comprising using a nitrile-based organic compound in association with the electrolyte of the battery, wherein the compound is C1 or C2 outlined below

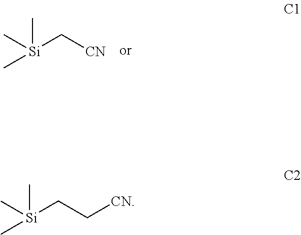

13. A method of improving the performance and safety of a Li-ion battery, comprising using a nitrile-based organic compound in association with the electrolyte of the battery, wherein the compound has a general formula IX outlined below 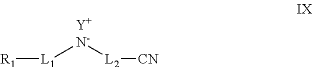
wherein:
R₁ is CN or CH₃;
L₁ and L₂ are each independently present or absent and are each independently a linker comprising alkyl, alkene and/or alkyne groups; and
Y is Na, K or Li; preferably Y is Na.

14. A method of improving the performance and safety of a Li-ion battery, comprising using a nitrile-based organic compound in association with the electrolyte of the battery, wherein the compound has a general formula X outlined below 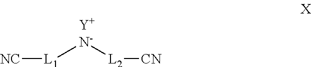
wherein:
L₁ and L₂ are each independently present or absent and are each independently a linker comprising one or more of alkyl, alkene and alkyne groups; and
Y is Na, K or Li; preferably Y is Na.

15. A method of improving the performance and safety of a Li-ion battery, comprising using a nitrile-based organic compound in association with the electrolyte of the battery, wherein the compound has a general formula XI outlined below 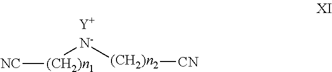
wherein:
n1 and n2 are each independently an integer from 0 to 10, or 0 to 6, or 0 to 3;
preferably at least one of n1 and n2 is 0, or both n1 and n2 are 0; and
Y is Na, K or Li; preferably Y is Na.

16. A method of improving the performance and safety of a Li-ion battery, comprising using a nitrile-based organic compound in association with the electrolyte of the battery, wherein the compound has a general formula D outlined below 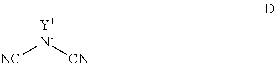
wherein Y is Na, K or Li; preferably Y is Na.

17. A method of improving the performance and safety of a Li-ion battery, comprising using a nitrile-based organic compound in association with the electrolyte of the battery, wherein the compound has a general formula D1 outlined below

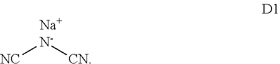

18. A compound having a general formula VII outlined below 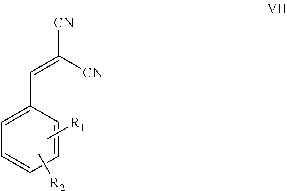
wherein R₁ and R₂ are each independently selected from the group consisting of H, alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, a cyano alkyl, a cyano alkene, a cyano alkyne, CN, NO₂, SO₂, COOH and acyloxycarbonyl; preferably selected from the group consisting of H, alkyloxy, halogen, halogeno alkyl, nitro and cyano; more preferably selected from the group consisting of H, halogen, nitro and cyano.

19. A compound having a general formula VIII outlined below 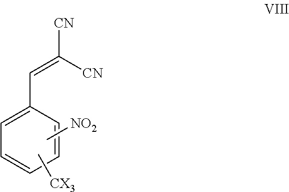
wherein X is a halogen atom; preferably X is F.

20. A compound of formula B4 outlined below

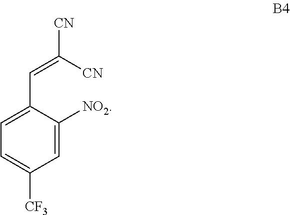 [SMILES: N#CC(C#N)=Cc1ccc(C(F)(F)F)cc1[N+](=O)[O-]]

21. A method of improving the performance and safety of a Li-ion battery, comprising using a nitrile-based organic compound in association with the electrolyte of the battery, wherein the compound as defined in any one of claims 18 to 20.

22. The method according to any one of claims 1 to 17 and 21, wherein the nitrile-based organic compound is added to the electrolyte; optionally an amount of the additive (nitrile-based organic compound) is between about 0.01 to about 5.0% wt, or about 0.01 to about 3.0% wt, or about 0.01 to about 1.0% wt, or about 0.05 to about 1.0% wt, or about 0.1 to about 1.0% wt, about 0.1 to about 0.8% wt, or about 0.1 to about 0.5% wt, or about 0.1 to about 0.3% wt, is 0.1% wt, or is 0.5% wt.

23. An electrolyte comprising a compound which is selected from the group consisting of: I, II, III, IV, A, B, A1, A2, A3, A4, B1, B2, B3, B4, B5, B6, B7, B8, V, VI, C, C1, C2, IX, X, XI, D, and D1 as defined in any one of method claims 1 to 17.

24. An electrolyte comprising the compound as defined in any one of claims 18 to 20.

25. A battery comprising the electrolyte as defined in claim 23 or 24.

26. An additive for an electrolyte for use in a Li-ion battery, comprising a compound which is selected from the group consisting of: I, II, III, IV, A, B, A1, A2, A3, A4, B1, B2, B3, B4, B5, B6, B7, B8, V, VI, C, C1, C2, IX, X, XI, D, and D1 as defined in any one of method claims 1 to 17.

27. An additive for an electrolyte for use in a Li-ion battery, comprising a compound as defined in any one of claims 18 to 20.

28. The method, electrolyte, battery or additive according to any one of claims 1 to 27, wherein the Li-ion battery is a battery wherein the cathode comprises a lithium-containing material.

29. The method, electrolyte, battery or additive according to any one of claims 1 to 27, wherein the Li-ion battery is a battery wherein the cathode comprises lithium cobalt oxide (LCO), lithium manganese oxide (LMO), lithium nickel oxide (LNO) and the like including olivines, lithium oxides, nickel manganese cobalt oxide (NMC).

30. The method, electrolyte, battery or additive according to claim 28 or 30, wherein the performance (capacity, reversibility) of the battery is improved.
